SEQUENCE LISTING (1 sequence)

SEQ ID NO: 1 - DNA, 1644 bp, Artificial Sequence
  codon optimized luciferase gene, effluc
atggaagatgccaagaacatcaagaaaggccctgcccccttctaccccctggaagatggcacagccggcgagcagctgcacaaggccatgaagagatacgccctggtgcccggcaccatcgccttcaccgacgcccacatcgaggtggacatcacctacgccgagtatttcgagatgagcgtgcggctggccgaggccatgaaacgctacggcctgaacaccaaccaccggatcgtggtgtgcagcgagaacagcctgcagttcttcatgcccgtgctgggcgccctgttcatcggcgtggccgtggcccctgccaacgacatctacaacgagcgggagctgctgaacagcatgggcatcagccagcccaccgtggtgttcgtgagcaagaagggcctgcagaaaatcctgaacgtgcagaagaagctgcccatcatccagaaaatcatcatcatggacagcaagaccgactaccagggcttccagagcatgtacaccttcgtgaccagccacctgccccctggcttcaacgagtacgacttcgtgcccgagagcttcgaccgggacaagaccatcgccctgatcatgaacagcagcggcagcaccggcctgcctaaaggcgtggccctgcctcaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaagagctgttcctgcggagcctgcaggactacaagatccagagcgccctgctggtgcccaccctgttcagctttttcgccaagagcaccctgatcgacaagtacgacctgagcaacctgcacgagatcgccagcggcggagcccccctgtccaaagaagtgggcgaagccgtcgccaagcggttccacctgcccggcatccggcagggctatggcctgaccgagaccacaagcgccattctgatcacccccgagggcgacgacaagcctggcgccgtgggcaaggtggtgcctttcttcgaggccaaggtggtggacctggacaccggcaagaccctgggcgtgaaccagcggggcgagctgtgcgtgaggggccccatgatcatgagcggctacgtgaacaaccccgaggccaccaacgccctgattgacaaggacggctggctgcacagcggcgacatcgcctactgggacgaggacgagcacttcttcatcgtggaccggctgaagagcctgatcaagtacaagggctaccaggtggccccagccgagctggaaagcatcctgctgcagcaccccaacatcttcgatgccggggtggccggactgcccgacgacgatgccggcgagctgcctgccgccgtggtggtgctggaacacggcaaaaccatgaccgagaaagaaatcgtggactacgtggccagccaggtgaccaccgccaagaaactgagaggcggcgtggtgtttgtggacgaggtgcccaagggcctgacaggcaagctggacgcccggaagatccgggagatcctgatcaaggccaagaagggcggcaagtga

(19) United States
(12) Patent Application Publication
(10) Pub. No.: US 2012/0278911 A1
(43) Pub. Date: Nov. 1, 2012

(54) TRANSGENIC ANIMAL OVEREXPRESSING LUCIFERASE AND PREPARATION METHOD THEREOF

(75) Inventors: Eun Young CHOI, Seoul (KR); Hye Won Youn, Seoul (KR)

(73) Assignee: SNU R&DB Foundation, Seoul (KR)

(21) Appl. No.: 13/456,400

(22) Filed: Apr. 26, 2012

(30) Foreign Application Priority Data
Apr. 26, 2011 (KR) 10-2011-0038999

(51) Int. Cl. A01K 67/027; C12N 5/10; C12N 15/63

(52) U.S. Cl. 800/18; 435/320.1; 435/354; 800/13

(57) ABSTRACT

The present disclosure provides a vector comprising a promoter and a luciferase gene having a nucleic acid sequence as disclosed in SEQ ID NO: 1; a fertilized egg transformed with the present vector; and a transgenic non-human animal overexpressing a luciferase gene from the vector and a method for preparing it. The vector and the animal of the present disclosure have a high expression rate for the luciferase gene, which confers high sensitivity for detection and thus useful for imaging analysis in a variety of research areas.

### CROSS-REFERENCE TO RELATED APPLICATIONS

This application claims the benefit of Korean Patent Application No. 10-2011-0038999, filed on Apr. 26, 2011 in the Korean Intellectual Property Office, the disclosure of which is incorporated herein by reference.

### BACKGROUND OF THE INVENTION

1. Field of the Invention

The present disclosure relates to a transgenic animal.

2. Description of the Related Art

Molecular imaging is a widely used technique in a variety of areas from in vitro and ex vivo imaging of organs and tissue cultures to the real time whole body in vivo imaging, and the demands keep increasing.

In molecular imaging, the various reporter genes for labeling proteins or cells have been widely developed since the visualization of proteins or cells is a key for the successful imaging.

Among them is a luciferase, which is an oxidative enzyme used in bioluminescence. One example is the fire fly luciferase from the firefly Photinus pyralis. It emits light over a wide spectral range with maximum at 560 nm and also emits a strong radiation at 600 nm, which makes them particularly useful for in vivo imaging. Firefly luciferase (Fluc) reacts specifically with D-luciferin in the presence of ATP, magnesium and oxygen to form luciferyl adenylate which in turn reacts with oxygen to form oxyluciferin, during which the light is released as the oxyluciferin returns to the ground state. The luciferin introduced in vivo by peritoneal injection reaches its maximum usually 20 min after the injection. Due to its stability and ease of use in obtaining the in vivo image, luciferin has been widely used for animal experiments. But its relatively slow reaction time is a drawback particularly when the images from the signal other than the luciferase also need to be obtained. The reaction takes about one hours during which images from other signals cannot be obtained.

Transgenic animals are a useful tool not only for the functional studies of genes in vivo, but also for studying the disease associated with a particular gene. Prior transgenic animals having luciferase genes have low sensitivity to the luciferin, which have resulted in the slow reaction time in vivo. Therefore there are demands for the new transgenic animal system overexpressing luciferase with high sensitivity.

### SUMMARY OF THE INVENTION

The present disclosure is to provide a transgenic non-human animal overexpressing luminescent protein with high sensitivity to luciferin.

Particularly the present disclosure provides a C57BL/6 strain transgenic mice, overexpressing a luminescent protein. The C57BL/6 strain has been known for its low efficiency for the production of transgenic mice compared to other strains such as FVB/N, B6D2F/1 and Swiss Webster (Auerbach et al., Strain dependent differences in the efficiency of transgenic mouse production, Transgenic Research 12 (2003): 59-69)

In one aspect, the present disclosure provides a vector pCAGGSeffluc comprising a promoter and a luciferase gene having a nucleic acid sequence as disclosed in SEQ ID NO: 1.

In other aspect, the present disclosure provides a fertilized egg transformed with the vector according to the present disclosure.

In still other aspect, the present disclosure provides a transgenic non-human animal transformed with the vector according to the present disclosure.

In still other aspect, the present disclosure provides a method for preparing a transgenic non-human animal overexpressing a luciferase protein comprising preparing a vector comprising a promoter and a luciferase gene having a nucleic acid sequence as disclosed in SEQ ID NO: 1; introducing the prepared vector to a fertilized egg; and implanting the fertilized egg to the uterine tube of a surrogate mother.

The transgenic non-human animal in accordance with the present disclosure have a high expression rate for the luciferase gene, leading to a high luminescence per cell. This enables the in vivo scanning of cells without sacrificing the animals. Thus the transgenic animal of the present disclosure provides a useful tool for disease research or cell tracking research, particularly in studying the development of cells and organisms, genetics, cancer biology, immunology, and/or cell and organ transplantation.

The foregoing summary is illustrative only and is not intended to be in any way limiting. Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic representation of the genetic map of pCAGGS used for the construction of the vector of the present disclosure.

FIGS. 2A through 2D are the FACS analysis results according to Example 6 of the present disclosure, indicating: (A) white blood cells; (B) splenocyte; (C) thymocyte; and (D) myelocyte.

FIG. 3 is the results from measuring the in vivo luminescent imaging in accordance with Example 7 of the present disclosure.

FIGS. 4A and 4B are the results from the skin graft experiment in accordance with Example 8 of the present disclosure, indicating: (A) the result using CD8 T cells (1×10⁶ cells) from B6 mouse mixed with CD4 T cells (1×10⁶ cells) from the transgenic mouse of the present disclosure expressing luciferase. The cells were intravenously injected into a male B6 mouse; (B) the result using CD4 T cells (1×10⁶ cells) from B6 mouse mixed with CD8 T cells (1×10⁶ cells) from the transgenic mouse of the present disclosure expressing luciferase. The cells were intravenously injected into a male B6 mouse.

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

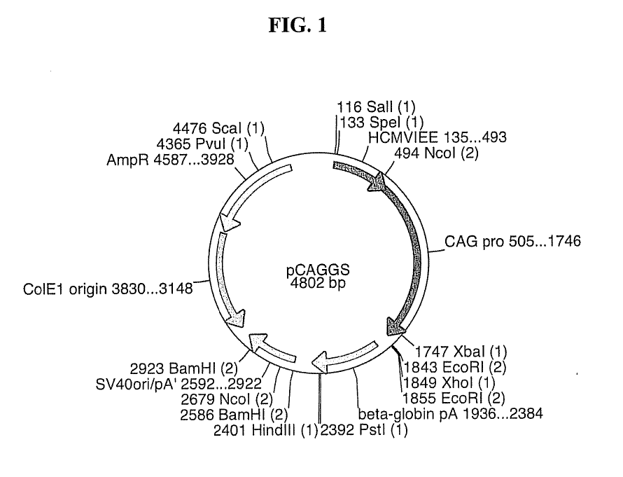

In one aspect, the present disclosure relates to a vector comprising a promoter and a luciferase gene with a DNA sequence as disclosed in SEQ ID NO: 1. The various backbone vectors may be used for the present disclosure for example as depicted in FIG. 1. In one embodiment the vector is pCAGGSeffluc.

The term “luciferase” as used herein refers to a generic term for the class of oxidative enzymes used in bioluminescence in a luciferin-luciferase (L-L reaction) reaction. The luciferase which may be used for the present disclosure is from various origins. One representative example is the firefly luciferase (Fluc) from the firefly Photinus pyralis and its derivatives. Commercially available luciferase genes such as Luc1, Luc2 (Promega, USA) or effluc may also be used for the present disclosure. Recently luciferase having a codon optimized sequence (SEQ ID NO: 1) has been developed and showed 100 times more sensitivity compare to the one with wild type. In one embodiment effluc is used. In other embodiment, the effluc is represented by a sequence as disclosed in SEQ ID NO: 1.

In other aspect the present disclosure relates to a transgenic non-human animal having a luciferase gene of SEQ ID NO:1.

The term “vector” as used herein refers to a gene construct comprising a control element operatively linked to a transgene or a heterologous gene encoded therein, which are expressed in cells where the vector is introduced. The vector which may be used for the present disclosure includes an expression control sequence such as a promoter, an operator, an initiation codon, a termination codon, a signal for polyadenylation and an enhancer in addition to other targeting signals such as a membrane targeting signal, a signal for secretion or a leader sequence. The control and/or targeting signals may be selected for the construction of a vector from the ones known in the art as desired by the skilled person in the art.

In one embodiment, the present disclosure provides a recombinant vector comprising a transgene having a nucleic acid sequence of SEQ ID NO:1, which is used to generated a fertilized egg transformed with the present vector.

In one embodiment, the promoters which may be used for the present disclosure include conventional promoters which are generally used to generate an expression vector for the expression of a heterologous gene. The promoters typically include ones for ubiquitous expression, conditional expression, or tissue specific expression. For the ubiquitous expression, the examples include, but are not limited to, SP6 promoter, T3 promoter, T7 promoter, or beta-actin promoter. For the tissue specific expression, the examples include, but are not limited to, ApoE, TTR (transthyretin) or albumin promoters for hepato specific expression, insulin promoter for pancrease beta cells, lck or CD2 promoters for T cells, and Camkinase 2 for neuronal cells. For the conditional expression, the example includes, but is not limited to, tetracyclin responding element.

In one embodiment, promoters for a ubiquitous expression are used to induce expression in all tissues or cell types. In other embodiment, beta-actin promoter is used. In still other embodiment, chicken beta actin promoter is used.

When the beta-actin promoter is used, it is preferable to use pCAGGS as a backbone vector for a strong expression for the encoded transgene, where the beta actin promoter is under the control of an enhancer from Cytomegalo Virus. Also the vector is stably maintained within a cell by integrating into the genome of a cell of the host. The genetic map of pCAGGS in accordance with one embodiment of the present disclosure is indicated as FIG. 1.

In other aspect, the present disclosure relates to a fertilized egg transformed with the present vector, particularly pCAGGSeffluc having a luciferase gene encoded therein with a nucleic acid sequence of SEQ ID NO: 1.

In one embodiment, fertilized eggs from various origins which are commonly used in the related field may be used for the present disclosure. In one preferred embodiment, fertilized eggs from mice are used.

The fertilized eggs may be produced by treating mice with gonadotropin to induce superovulation. The hormone which may be used includes, but is not limited to, pregnant mare's serum gonadotropin (PMSG), or human chorionic gonadotropin (HCG) or a combination thereof.

In one embodiment, the fertilized egg is derived from C57BL/6 strain, particularly the fertilized egg deposited on Apr. 1, 2011, with the Korean Collection for Type Cultures under the deposit number KCTC11912BP.

Any suitable methods known in the art to introduce pCAGGS comprising a luciferase gene, i.e., pCAGGSeffluc to a fertilized egg may be used. The examples include, but are not limited to, microinjection technique to introduce a transgene into a pronucleus of a fertilized egg shortly after a fertilization; a stem cell insertion technique where a transgene is introduced to an embryonic stem cell followed by a implantation of the cell into an embryo at the blastocyst stage; a retrovirus insertion technique where retroviruses are used as carriers to deliver a transgene into a fertilized egg; and a sperm mediated gene transfer technique where a transgene is injected into the testis of a male animal and the sperms are used to fertilize ova. In one embodiment, the microinjection technique is used to generate an ovum derived from a transgenic mouse of C57BL/6 strain transformed with a luciferase gene having a nucleic acid sequence as disclosed in SEQ ID NO: 1.

In other aspect, the present disclosure relates to a non-human transgenic animal transformed with the vector in accordance with the present disclosure.

The term “transgene” as used herein refers to a desired DNA sequence or a gene introduced into a vector and/or the animal's genome, including but not limited to genes or DNA sequences which may not normally be present in the genome, genes which are present, but not normally transcribed and translated (“expressed”) in a given genome, or any other genes or DNA sequences which one desires to introduce into the genome. This may include genes which may normally be present in the non-transgenic genome but which one desires to have altered in expression, or which one desires to introduce in an altered or variant form.

The term “transgenic animal” as used herein refers to a non-human animal with a transgene as described above with a trait changed due to a heterologous recombinant gene integrated into its genome. The examples of non-human animal include mammals, particularly mammals which is able to develop a disease similar to the ones found in human so that one may use it as a model to study etiology and pathogenesis of a disease of interest. The suitable animals include vertebrates having an internal structure, an immune system and/or a body temperature similar to those of humans and being capable of developing a high blood pressure, cancer or immune deficiency. By way of example, mammals such as sheep, pigs, goats, camels, antelope, dogs, rabbits, mice, rats, guinea pigs, or hamsters may be used.

In one embodiment, mice are used. Mice are widely used for the disease research because they are very fertile and genetically homogenous in addition to the ease of control and maintenance. A various types of mice which is able to develop symptoms or disease which is same or similar to humans have been developed. For example, C57BL/6 mice strain, the genetic background of which, have been completely identified is widely used for research in genetics, immune and cancer biology, development, organ transplantation and the like.

In certain aspects, the present disclosure relates to a method for preparing a transgenic non-human animal overexpressing a luciferase protein comprising: preparing a vector comprising a promoter and a luciferase gene having a nucleic acid sequence as disclosed in SEQ ID NO: 1; introducing the prepared vector into a fertilized egg; and implanting the fertilized egg to the uterine tube of a foster mother.

In one embodiment, the vector may be prepared by conventional methods known in the art, for example, by obtaining a luciferase gene having a nucleic acid sequence as disclosed in SEQ ID NO: 1 by PCR amplification and ligating the amplified product into a suitable vector of interest.

In other embodiment, the transgenic animal is from a C57BL/6 mouse strain. The transgenic mice may be generated by using a fertilized egg, which is obtained by treating female mice with a gonadotropin to induce superovulation and allowing the female mice to mate with male mice. The gonadotropin as described above may be used.

In other embodiment, the vector which may be used to generate a non-human transgenic animal is pCAGGSeffluc. For injection into a fertilized egg, not the whole vector but only the part that contains the elements necessary for the expression of the gene of interest may be used. In one embodiment, a 4.3 kb fragment of pCAGGSeffluc is used for the injection after digestion of the present vector with proper enzymes.

Various methods to introduce DNA of interest into a fertilized egg or embryo as disclosed above may be used, which include without limitation microinjection, stem cell insertion technique, retroviral insertion technique and sperm-mediated gene transfer technique. In one embodiment, the luciferase gene of SEQ NO ID: 1 of the present disclosure is introduced using microinjection technique to generate a transgenic fertilized egg from C57BL/6 mice

The transgenic mice may be generated by implanting the transgenic fertilized egg into the uterine of a foster mouse.

The transgenic mice as prepared above showed the increased level of luciferase expression in all the organs examined, which enables the detection of luminescence within less than 10 min after the peritoneal injection of the substrate luciferin.

The present disclosure is further explained in more detail with reference to the following examples. These examples, however, should not be interpreted as limiting the scope of the present invention in any manner.

### EXAMPLES

### Example 1

### Construction of a Recombinant Vector pCAGGSeffluc Comprising a Chicken Beta-Actin Promoter and a Codon-Optimized Luciferase Gene

For the construction of the plasmid, PCR was performed on a template pDONR222-eGFP-codon-optimized luciferase DNA (Dr. B. A. Rabinovich of University of Texas) with a sense primer: 5′-TCTAGAATGGAAGATGCCAAGAACATCAAG-3′; and an antisense primer: 5′-CTCGAGCTACTTGCCGCCCTTCTTGGC-3′ using Taq polymerase (Takara, Japan) and 34 cycles of denaturation at 95° C. for 1 min; annealing at 60° C. for 1 min; and extension at 72° C. for 1 min. The amplified products were then electrophoresed on a 1% agarose gel and the band corresponding to 1.644 kb of codon-optimized luciferase DNA was excised and purified using gel extraction kit according to the manufacturer's instruction (Intron, Korea). The purified product was then cloned into a pGem-T easy vector (Promega, USA) and T4 DNA ligase (Koschem, Korea). The cloned plasmid containing the fragment in correct direction was selected and confirmed by digesting the plasmid with EcoRI (Koschem) and the cloned codon-optimized Luciferase was further confirmed by the sequence analysis (Macrogen, Korea).

To construct pCAGGSeffluc, the codon-optimized luciferase gene was cloned to a pCAGGS vector under the control of a chicken beta-actin promoter. The pGemT easy vector having codon-optimized luciferase gene as prepared above and pCAGGS were treated with XhoI and Xba I (Koschem). The digested products were then electrophoresed on a 1% agarose gel and the bands corresponding to the expected sizes were excised and purified using a DNA gel extraction kit (Intron) to obtain a linearized vector and the codon-optimized luciferase gene. The obtained fragments were then mixed and ligated with T4 DNA ligase by incubating overnight at 16° C. to construct a recombinant product of the codon-optimized luciferase gene linked to the chicken beta actin promoter, which was confirmed by sequencing (Macrogen).

### Example 2

### Preparation of Chicken Beta-Actin Promoter Linked to Codon-Optimized Luciferase for the Generation of Transgenic Mouse

For preparing large quantities of DNA for microinjection, pCAGGSeffluc as prepared in Example 1 was digested with SalI and BamHI to generate three fragments of 4.304 kb, 1.998 kb and 0.332 kb in size. Of those, 4.304 kb fragment containing the codon-optimized luciferase gene linked to the promoter was recovered, which was then dialyzed in 1×TE buffer (10 mM Tris-HCl, 0.1 mM EDTA, pH7.4). The concentration was then adjusted to 2-4 ng/μl and used for the microinjection as described below.

### Example 3

### Preparation of Fertilized Eggs, Microinjection and Implantation Thereof

Female C57BL/6 mice were superovulated by treating them with each of 5 IU of Pregnant Mares Serum Gonadotropin (Serotropin, Japan) and HCG (Human Chorionic Gonadotropin: Sigma, USA) once with a 48 hour interval. Then the mice were then allowed to mate with male C57BL/6 mice. Next day, mice with a copulation plug were selected and sacrificed by cervical dislocation followed by the removal of the uterine tubes. The cumulus cell mass was harvested from the excised uterine tubes, and the cells were treated with 300 unit/ml of hyaluronidase solution for 2-3 min followed by a centrifugation at 15,000 rpm for 3 min.

Then the DNA as prepared in Example 2 was used for microinjection into the fertilized egg as prepared above. The resulting embryo was then allowed to grow up to 2 cell stage in a CO₂ incubator, some of which were then used for the deposition (KCTC 11912BP) and for implanting into a foster mother (ICR strain)

### Example 4

### Confirmation of the Successful Transformation

To confirm that the codon-optimized luciferase transgene was successfully introduced and expressed in the progenies generated in Example 3, the tail was cut from the mice at about 0.5 cm in length and the genome from the excised tail was extracted by treating the tail with a solution containing proteinase K. The extracted genome was then used as a template DNA in a PCR reaction using 5′-TTGCCTTTTATGGTAATCGTGCGAGA-3′ as a sense primer and 5′-TATCTCTTCATGGCCTTGTGCAGCT-3′ as an antisense primer.

### Example 5

### Luciferase Reporter Assay

The peripheral blood was obtained from the ophthalmic vein of the transgenic mice expressing codon-optimized luciferase as prepared above. The blood obtained was then mixed with ACK lysis solution (150 mM NH₄Cl, 1 mM KHCO₃, 0.1 mM Na₂EDTA) to remove red blood cells and obtain peripheral lymphocytes. The prepared cells were then mixed with 100 μl of reporter lysis buffer (Promega) and the mixture was incubated on ice for 10 min followed by a centrifugation at 13,000 rpm for 1 min. The supernatant was collected and 60 μl of which was added to a well of a 96 well plate (Falcon, USA) followed by the addition of substrate A and B (Promega) to the well. The luciferase activity was measured using a luminometer (Perkin Elmer, USA) and Wallac 1420 program.

The results are shown in Table 1 below.

| Plate | Repeat | Well | Type | Time | 1 sec(CPS) | No | Remark |
|---|---|---|---|---|---|---|---|
| 1 | 1 | B01 | M | 00:00:10.98 | 242 | | pCAGGS in 293T |
| 1 | 1 | B02 | M | 00:00:12.25 | 38533 | | pCAGGSeffluc in 293T |
| 1 | 1 | C01 | M | 00:00:15.06 | 25200 | #1 | Tg-F0#15 F1 |
| 1 | 1 | C02 | M | 00:00:16.33 | 18977 | #3 | Tg-F0#15 F1 |
| 1 | 1 | C03 | M | 00:00:17.60 | 12446 | #5 | Tg-F0#15 F1 |
| 1 | 1 | C04 | M | 00:00:18.88 | 17107 | #6 | Tg-F0#15 F1 |
| 1 | 1 | C05 | M | 00:00:20.15 | 2405 | #7 | Tg-F0#16 F1 |
| 1 | 1 | C06 | M | 00:00:21.43 | 73 | #22 | Tg-F0#116 F1 |
| 1 | 1 | C07 | M | 00:00:22.70 | 88 | #25 | Tg-F0#09 F1 |
| 1 | 1 | C08 | M | 00:00:23.98 | 126 | #29 | Tg-F0#09 F1 |
| 1 | 1 | C09 | M | 00:00:25.25 | 120 | #30 | Tg-F0#19 F1 |
| 1 | 1 | C10 | M | 00:00:26.53 | 84 | #31 | Tg-F0#19 F1 |
| 1 | 1 | C11 | M | 00:00:27.80 | 101 | B6 | Negative control |

As indicated in Table 1, among founders, progeny of Tg-F0#15 (F1) had a luciferase activity superior to that of other transgenic mice. Therefore the embryos of Tg-F0#15 were deposited with the international depository authority, Korean Collection for Type Cultures under the Budapest Treaty on Apr. 1, 2011 and the accession number KCTC11912BP was given.

### Example 6

### Flow Cytometer Analysis

The transgenic mice expressing codon-optimized luciferase gene as prepared above were sacrificed, and the spleen, thymus and bone marrow were removed and kept in 1×PBS at 4° C. The single cell suspension was prepared from each of the excised organs and resuspended in 1×PBS and the cell numbers were counted.

The cells from each organ as prepared above were mixed with flow cytometer buffer solution (FACS buffer solution: 0.1% NaN3, 0.1% BCS, 1×PBS) and anti-mouse CD4 (clone: GK1.5), anti-mouse B220 (clone: RA3-6B2), anti-mouse CD8 (clone: 53-6.7), anti-mouse Mac1 (clone: M1/70), anti-mouse Gr1 (clone: RB6-8C5), anti-mouse CD11c (clone: N418), anti-mouse CD3 (clone: 145-2C11) were added thereto followed by an incubation at 4° C. for 30 min. All the antibodies used in the experiment were obtained from eBioscience (USA). The cells were then washed once with 3 ml of FACS buffer and analyzed on FACSCalibur (BD Biosciences, USA) with CellQuest software.

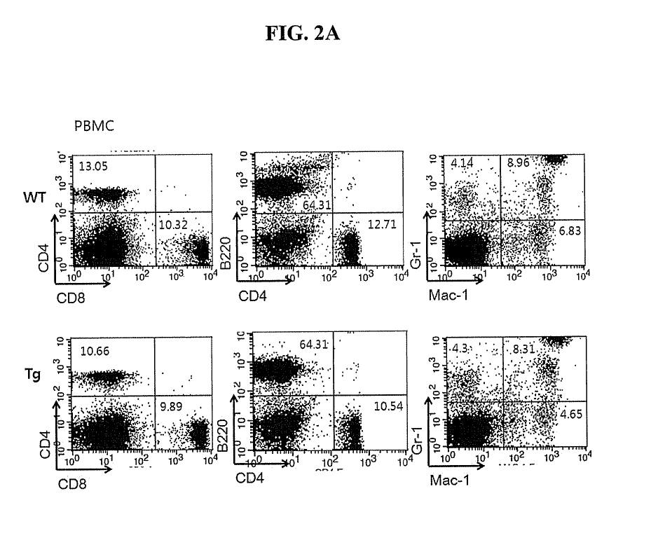

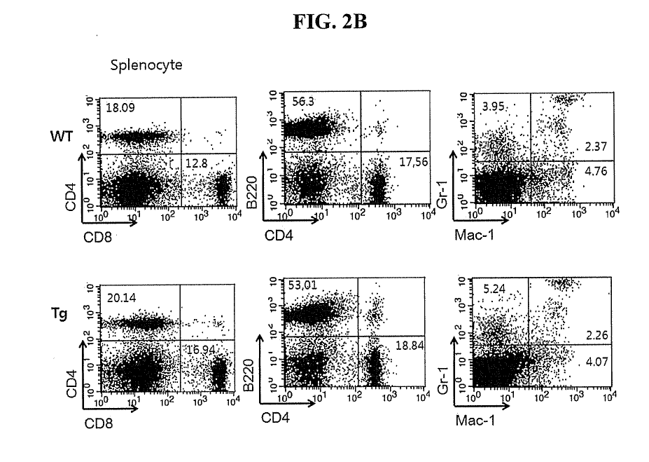

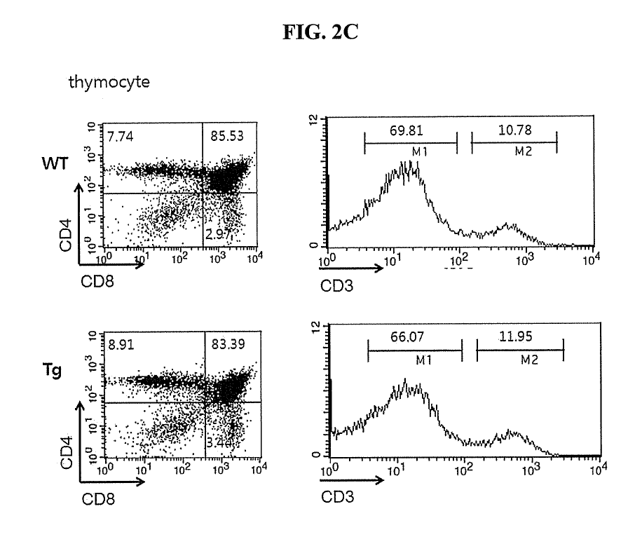

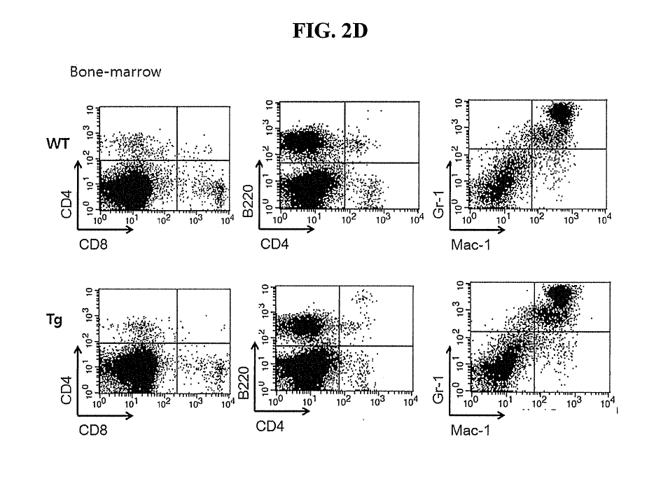

The results are shown in FIGS. 2A to 2D, which each indicates the results using white blood cells; splenocyte; thymocyte; and myelocyte, respectively. No difference was found between the control mouse and the transgenic mouse in the development profile of immune cells. This indicates that the transgenic mice according to the present disclosure can be useful as a tool for studying immunological disease.

### Example 7

### Bioluminescence Measurement

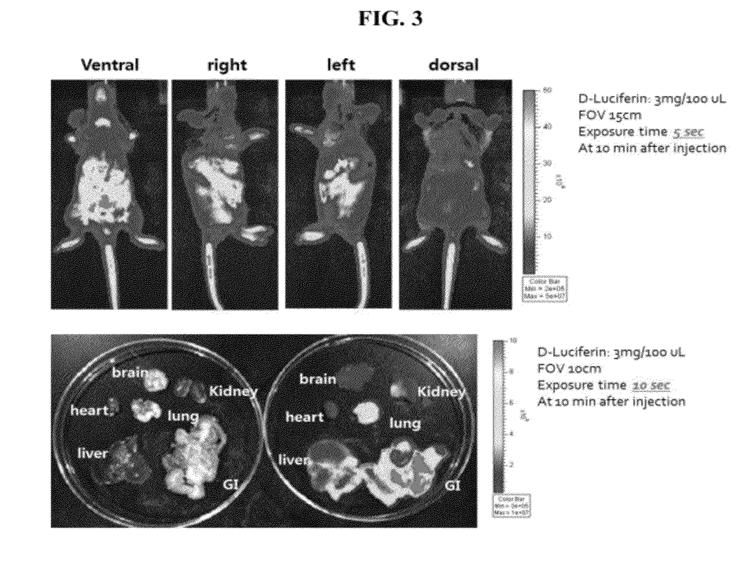

A 3 mg/100 μl of D-luciferin was peritoneally injected into the transgenic mice expressing codon-optimized luciferase as prepared above. After 10 min of the injection, the mouse was placed on the IVIS-100 (Xenogene, USA). The IVIS-100 was set up as gray-scale image and bioluminescence FOV 10 cm, f16 f/stop, medium binning and the exposure time was set to 1 sec to 5 min. The images from each of dorsal, ventral, left and right sides of the transgenic mouse were taken. The results are shown in FIG. 3.

As indicated in FIG. 3, the strong luminescence was observed in all the organs examined within 10 min after the D-luciferin injection.

### Example 8

### Skin Grafting

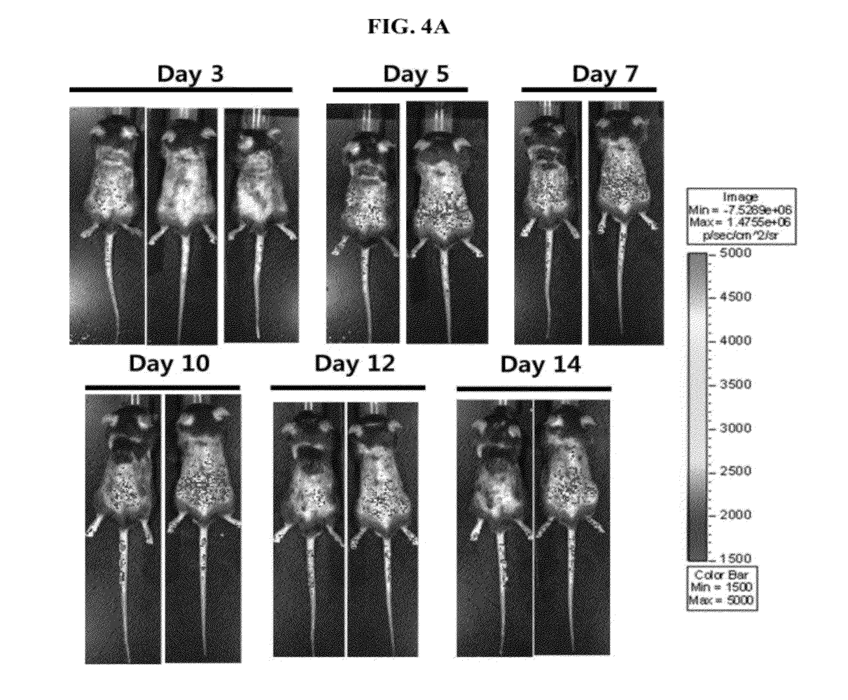

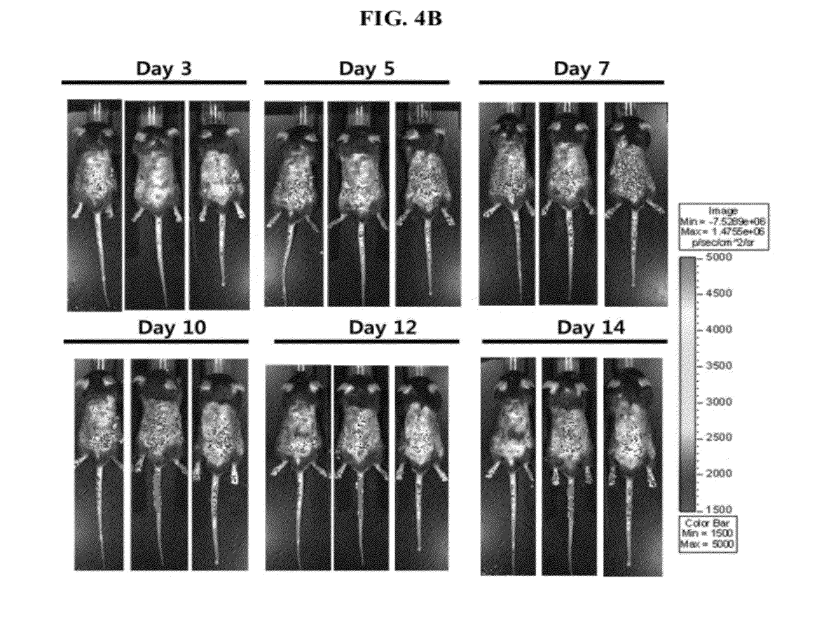

The single cell suspension was obtained from each of a male B6 mice (Charles River, USA) C57BL/6 mice (Charles River) and the transgenic mice expressing codon-optimized luciferase. CD4 T cells and CD8 T cells were isolated from the suspension using MACS (Miltenyi biotech, USA). The isolated CD4 T cells (1×10⁶) from B6 mice and CD8 T cells (1×10⁶) from the transgenic mice were mixed and injected intravenously into a male 86 mouse. Also the isolated CD8 T cells (1×10⁶) from B6 mice and CD4 T cells (1×10⁶) from the transgenic mice were mixed and injected intravenously into a male B6 mouse. 24 hours after the injection, the B6 male mouse was anesthetized with Avertin® (Sigma Aldrich, USA) and four skin excisions of 0.5 cm in length were made on the tail with a regular interval without damaging the vein. The same procedures were done with a BALB.B mouse which was used as a skin donor. The skin fragment from the donor (BALB.B) was grafted onto the three excisions made on the recipient (B6). The remaining one excision site was self-grafted. The grafted area of the tail was covered with gauze and fixed with adhesive tape, which was then placed in a glass tube. The results are shown in FIGS. 4A and 4B.

FIG. 4(A) is the result using CD8 T cells (1×10⁶ cells) from 86 mouse mixed with CD4 T cells (1×10⁶ cells) from the transgenic mouse of the present disclosure expressing luciferase. The prepared cells were injected intravenously into a male B6 mouse; (B) the result using CD4 T cells (1×10⁶ cells) from 86 mouse mixed with CD8 T cells (1×10⁶ cells) from the transgenic mouse of the present disclosure expressing luciferase. The prepared cells were injected intravenously into a male 86 mouse.

As indicated in FIG. 4A, after the skin graft, CD4 T cells were moved to and proliferated in the spleen and the lymph nodes (before day 10), after which they moved to the grafted skin from the donor (BALB.B) (day 10) but did not moved to the self-grafted skin fragment (B6). In case of CD8 T cells, the migration of CD8 T cells to the spleen and the lymph nodes was confirmed after the skin graft (before day 10), and the strong signal from CD8 T cells migrated to the grafted skin from the donor was detected (day 10 to day 14). It indicates that CD8 T cells also did not migrated to the self-grafted skin (FIG. 4B).

With respect to the use of substantially any plural and/or singular terms herein, those having skill in the art can translate from the plural to the singular and/or from the singular to the plural as is appropriate to the context and/or application.

The various singular/plural permutations may be expressly set forth herein for sake of clarity. Although a few embodiments of the present invention have been shown and described, it would be appreciated by those skilled in the art that changes may be made in this embodiment without departing from the principles and spirit of the invention, the scope of which is defined in the claims and their equivalents.

1. A pCAGGSeffluc vector comprising a promoter and a luciferase gene having a nucleic acid sequence as disclosed in SEQ ID NO: 1.

2. The vector according to claim 1, wherein the promoter is for a ubiquitous expression, a conditional expression or a tissue specific expression.

3. The vector according to claim 2, wherein the promoter is for a ubiquitous expression.

4. The vector according to claim 3, wherein the promoter for the ubiquitous expression is a beta-actin promoter.

5. The vector according to claim 4, wherein the beta-actin promoter is a chicken beta-actin promoter.

6. A fertilized egg transformed with the vector as disclosed in claim 1.

7. The fertilized egg according to claim 6, wherein the egg is deposited with the Korean Collection for Type Cultures under the deposit No: KCTC11912BP.

8. A transgenic non-human animal transformed with the vector as disclosed in claims 1.

9. The transgenic animal according to claim 8, wherein the animal is produced by implanting a fertilized egg transformed with the vector to the uterine of a foster.

10. The transgenic animal according to claim 8, wherein the animal overexpress a luciferase gene having a nucleic acid sequence as disclosed in SEQ ID NO: 1.

11. The transgenic animal according to claim 8, wherein the animal is a mouse of C57BL/6 strain.

12. The transgenic animal according to claim 6, wherein the C57BL/6 strain mouse is used as a research tool for studying cell development, cell differentiation, cell migration, cell proliferation, cell or organ transplant, genetics, or for immune and cancer research.

13. A method for preparing a non-human transgenic animal overexpressing a luciferase protein comprising: preparing a vector comprising a promoter and a luciferase gene having a nucleic acid sequence as disclosed in SEQ ID NO: 1; introducing the prepared vector to a fertilized egg; and implanting the fertilized egg to the uterine tube of a foster.

14. The method of claim 13, wherein the introduction of the vector is performed by a technique selected from a group consisting of a microinjection technique, a stem cell insertion technique, a retroviral insertion technique and sperm-mediated gene transfer technique.

15. The method according to claim 13, wherein the animal is a mouse of C57BL/6 strain.

16. The method according to claim 15, wherein the fertilized egg is prepared by treating a female mouse of C57BL/6 strain with a gonadotropin to induce superovulation followed by mating the female mouse with a male mouse of C57BL/6 strain.

17. The method according to claim 16, wherein the gonadotropin is a pregnant mare's serum gonadotropin (PMSG) or human chorionic gonadotropin (HCG) or a combination thereof.
